(19) United States
(12) Patent Application Publication
(10) Pub. No.: US 2006/0051403 A1
(43) Pub. Date: Mar. 9, 2006

(54) Microprojection array with improved skin adhesion and compliance

(76) Inventors: James Matriano, Mountain View, CA (US); Michel J.N. Cormier, Mountain View, CA (US); Peter Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/222,297

(22) Filed: Sep. 7, 2005

Related U.S. Application Data
(60) Provisional application No. 60/608,280, filed on Sep. 8, 2004.

(51) Int. Cl. A61F 13/02; A61L 15/16

(52) U.S. Cl. 424/448; 604/500

(57) ABSTRACT

A transdermal delivery system having a microprojection array with one or more voids configured to allow an adhesive backing to attach to a user's skin and, hence, improve retention of the system thereto. Radial voids can be configured to enhance compliance of the array to conform more readily to non-flat areas of the skin.

### CROSS-REFERENCE TO RELATED APPLICATION

This application claims benefit of U.S. Provisional Application No. 60/608,280, filed Sep. 8, 2005.

### FIELD OF THE PRESENT INVENTION

The present invention relates generally to active agent delivery systems and methods. More particularly, the invention relates to transdermal delivery of active agents via microprojection arrays configured to exhibit improved skin retention.

### BACKGROUND ART

As is well known in the art, transdermal delivery provides for a method of administering active agents to a host that would otherwise need to be delivered via hypodermic injection or intravenous infusion. The word “transdermal”, as used herein, is generic term that refers to delivery of an active agent through the skin to the local tissue, particularly the dermis and epidermis, or systemic circulatory system, without substantial cutting or penetration of the skin, such as cutting with a surgical knife or piercing the skin with a hypodermic needle. Transdermal agent delivery includes delivery via passive diffusion as well as active delivery based on external energy sources, such as electrical (iontophoresis, for example) and ultrasound (phonophoresis, for example).

As is also well known in the art, the transdermal drug flux is dependent upon the condition of the skin, the size and physical/chemical properties of the drug molecule, and the concentration gradient across the skin. Because of the low permeability of the skin to many drugs, passive transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum, particularly to hydrophilic and high molecular weight drugs and macromolecules, such as proteins, naked DNA, and viral vectors.

To overcome the difficulties presented by delivering larger molecules by passive diffusion through the skin, there have been many techniques and systems developed to mechanically penetrate or disrupt the outermost skin layers. The goal of such approaches is to create pathways into the skin in order to enhance the amount of agent being transdermally delivered. Such physical methods of permeation enhancement include sandpaper abrasion, tape stripping and bifurcated needles. While these techniques increase permeability, it is difficult to predict the magnitude of their effect on drug absorption. Laser ablation, another physical permeation enhancer, may provide more reproducible effects, but it is currently cumbersome and expensive.

Transdermal delivery systems and apparatus, which employ tiny skin piercing elements to enhance transdermal agent delivery, are disclosed in U.S. Pat. Nos. 5,879,326, 3,814,097, 5,250,023, 3,964,482, U.S. Pat. Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all of which are incorporated by reference herein in their entirety. The piercing elements (or microprojections) disclosed in these references comprise various shapes and generally extend perpendicularly from a thin, flat member, such as a pad or sheet. The piercing elements are also typically extremely small, some having a microprojection length of only about 25-400 μm and a microprojection thickness of only about 5-50 μm.

The disclosed delivery systems have been found extremely effective in administering active agents to a user and, hence, have been substantially embraced by the industry. Significant effort and resources have thus been, and continue to be, expended toward developing microprojection array patch technology and agent formulations associated therewith to increase the number and type of agents that can be transdermally delivered through the skin.

One key area of microprojection patch technology that has been developed is coated microprojection systems, wherein an active agent formulation is coated on the microprojections. Illustrative is the microprojection systems disclosed in U.S. patent application Ser. Nos. 10/045,842, 10/127,108, 10/637,909 and 60/473,273, which are fully incorporated by reference herein. As set forth in the noted applications, upon application of the microprojection member or patch, the microprojections create superficial pathways through the stratum corneum, whereby the coating is dissolved by interstitial fluid and the agent is delivered into the dermis, epidermis and deeper tissue.

Despite the suitability of microprojection arrays to deliver active agents, there are several issues associated with the prior art systems that must be addressed. A major issue is the retention (or adherence) of the microprojection array to the user's skin. Indeed, in many instances, reproducibility and optimal delivery of agents coated on microprojection arrays is largely dependant on the use of retention features on the array.

Attempts to maximize retention of microprojection arrays on the skin have largely centered around the use of adhesives. For example, several systems employ an adhesive around the periphery of the array to secure the patch to the skin for delivery. However, in such systems skin retention is significantly reduced at the center of the microprojection array due to the location of the peripheral adhesive.

Since microprojection arrays are generally formed by etching the microprojections from a uniform sheet of material and subsequently bending the microprojections to an orientation about perpendicular to the sheet, a plurality of openings are formed in the sheet corresponding to each microprojection. Attempts have thus been made to use an adhesive backing that would be exposed through these openings to aid in skin retention. However, given the desired size of the microprojections, it has been found that these openings are generally too small to provide adequate exposure of the adhesive and consequently have not significantly improved retention.

Yet other prior art attempts to improve skin retention have involved configuring the tips of the microprojections to provide barbs or hooks to facilitate anchoring the array in the skin. Examples of such configurations are set forth in U.S. Pat. Nos. 6,050,988 and 5,312,456, the disclosures of which are incorporated by reference herein in their entirety. However, such tip configurations can increase the risk of injury to the uppermost layers of the skin during removal of the array.

Accordingly, it is an object of the invention to provide a microprojection array having improved skin retention.

It is a further object of the invention to provide a microprojection array having substantially uniform adhesion characteristics across the surface of the array.

It is yet another object of the invention to provide a microprojection array having increased surface area while maintaining adequate skin retention.

Another object of the invention is to provide a microprojection array having enhanced flexibility and improved compliance.

### SUMMARY OF THE INVENTION

In accordance with the above objects and those that will be mentioned and will become apparent below, the delivery system for transdermally delivering an active agent in accordance with this invention includes a microprojection array with a plurality of stratum corneum-piercing microprojections and at least one void in the microprojection array. Preferably, the array includes an adhesive backing that communicates through the void.

In one embodiment of the invention, the void has a surface area greater than approximately 0.2 mm². Preferably, the void has a surface area of at least approximately 3 mm². In another aspect of the invention, the size of the void is at least approximately 2 mm as measured across its widest length.

Preferably, the microprojection array includes a plurality of voids that are adapted to communicate with the adhesive backing.

Preferably, the system has a total void area in the range of approximately 5-50% of the total microprojection array area. More preferably, the total void area is in the range of approximately 10-30% of the total microprojection array area.

In certain embodiments, the microprojection array has a total area greater than approximately 1 cm². Preferably, the total area is up to approximately 5 cm².

In other embodiments, the microprojection array has at least one radial void that preferably extends from a central portion of the microprojection array to the periphery. In at least one embodiment, the radial void divides the array into at least two discrete subunits.

Preferably, the microprojection array comprising a radial void has a total area greater than approximately 3 cm².

In one embodiment of the invention, the adhesive backing is compliant. In one aspect of the noted embodiment, the compliant backing is perforated.

The invention also comprises methods of delivering an active agent by (i) providing a delivery system having a microprojection member that includes a plurality of microprojections, at least one void, an agent-containing coating on at least one of the microprojections and an adhesive backing, and (ii) applying the microprojection member to a user's skin, whereby the adhesive backing adheres to the skin through the void. In at least one embodiment, the delivery system includes a radial void and the step of applying the microprojection member comprises applying the member to a domed, non-flat region of the skin.

### BRIEF DESCRIPTION OF THE DRAWINGS

FIG. 1 is a schematic bottom view of a prior art microprojection array;

FIG. 2 is cross-section view of the microprojection array shown in FIG. 1

FIG. 3 is a partial perspective view of a microprojection array having a coating on the microprojections;

FIG. 4 is a schematic bottom view of a microprojection array of the invention having a void;

FIG. 5 is cross-section view of the microprojection array shown in FIG. 4;

FIGS. 6-9 are alternative configurations of microprojection arrays of the invention having voids; and

FIGS. 10-16 are alternative configurations of microprojection arrays of the invention having radial voids.

### DETAILED DESCRIPTION OF THE INVENTION

Before describing the present invention in detail, it is to be understood that this invention is not limited to particularly exemplified array and/or void sizes, shapes and orientations, materials, methods or structures as such may, of course, vary. Thus, although a number of array and/or void sizes, shapes and orientations, materials and methods similar or equivalent to those described herein can be used in the practice of the present invention, the preferred array and/or void sizes, shapes and orientations, materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a void” includes two or more such voids.

### Definitions

The terms “transdermal”, “intradermal”, “intracutaneous”, “intradermally”, “intracutaneously”, “transcutaneous”, “transdermally” and “transcutaneously” are used interchangeably herein to mean the delivery of an agent into and/or through the skin for local or systemic therapy. The noted terms thus mean and include intracutaneous, intradermal and intraepidermal delivery of an active agent into and/or through the skin via passive diffusion as well as energy-based diffusional delivery, such as iontophoresis and phonophoresis.

The term “transdermal flux”, as used herein, means the rate of transdermal delivery.

The term “active agent”, as used herein, means any therapeutic agent, drug, compound, molecule or the like having biological, pharmacological, diagnostic or therapeutic activity.

The term “active agent” also includes an “antigenic agent” and/or “vaccine”, as used interchangeably herein, which refer to a composition of matter or mixture containing an immunologically active agent that is capable of triggering a beneficial immune response when administered in an immunologically effective amount. The terms “antigenic agent” and “vaccine” thus include, without limitation, protein-based vaccines, polysaccharide-based vaccine, nucleic acid-based vaccines, viruses and bacteria.

The term “biologically effective amount” or “biologically effective rate”, as used herein, refers to the amount or rate of the active agent needed to stimulate or initiate a beneficial result.

The term “microprojections”, as used herein, refers to piercing elements that are adapted to pierce or cut through the stratum corneum into the underlying epidermis layer, or epidermis and dermis layers, of the skin of a living animal, particularly, a mammal and, more particularly, a human. According to the invention, the microprojections can be formed in different shapes, such as needles, hollow needles, blades, pins, punches, and combinations thereof.

In one embodiment of the invention, the microprojections have a projection length of at least 100 μm. In another embodiment of the invention, the microprojections have a projection length less than 1000 μm. In a further embodiment, the microprojections have a projection length of less than 500 μm, more preferably, less than 250 μm. The microprojections typically have a width and thickness of about 5 to 50 μm. The microprojections also preferably have a width of about 75 to 500 μm.

The terms “microprojection member” and “microprojection array”, as used herein, generally connote a plurality of microprojections arranged in an array for piercing the stratum corneum. According to the invention, the microprojection member can be formed by etching or punching a plurality of microprojections from a thin sheet and folding or bending the microprojections out of the plane of the sheet to form a configuration such as that shown in FIG. 3 and described in U.S. Pat. No. 6,083,196, which is hereby incorporated by reference in its entirety. The microprojection member can also be formed in other known manners, such as by forming one or more strips having microprojections along an edge of each of the strip(s), as disclosed in U.S. Pat. No. 6,050,988, which is hereby incorporated by reference in its entirety. Other microprojection arrays, and methods of making same, are disclosed in U.S. Pat. Nos. 5,879,326 and 5,983,136.

Conventional methods for forming microprojection arrays generally comprise controlled manufacturing processes. For example, a typical method involves generating an computer-aided design corresponding to the desired microprojection array that is used with photo/chemical etching to form the array. First, a thin laminate resist is applied on a sheet of titanium about 30 μm thick. The resist is contact-exposed using a mask with the desired pattern and then developed. The sheet is then etched using acidic solutions to form the microprojections. After etching, the microprojections are folded generally perpendicular to the sheet using a forming tool.

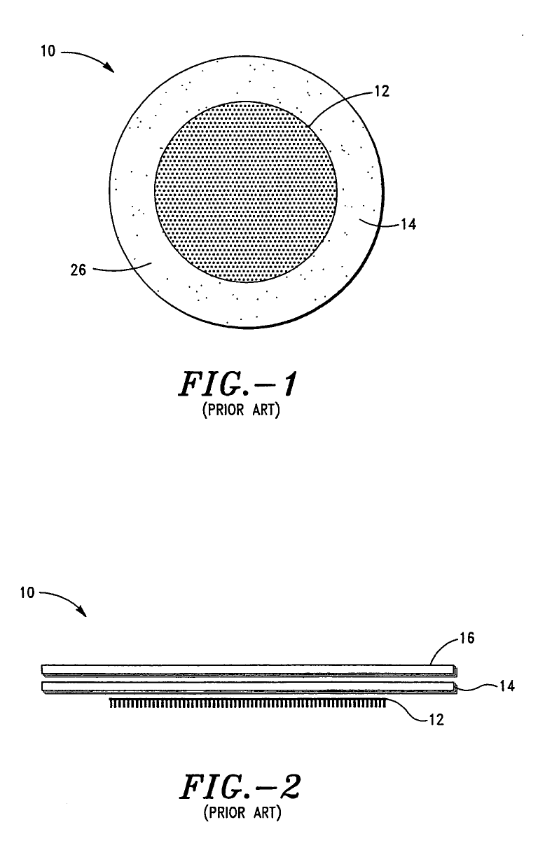

As shown in FIG. 1, a prior art microprojection array patch 10 presents a uniform dispersion of microprojections across the working area of microprojection array 12. An adhesive layer 14 is deposited on the array and covered with a backing 16, as shown in cross-section in FIG. 2. FIG. 3 shows a detail view of the microprojection array 12, with stratum corneum-piercing microprojections 18 having a uniform coating 20 of active agent or a pattern coating 22 of active agent on a specific portion of the microprojection. Microprojections 18 are shown with a barb to aid in retaining the device once embedded in the skin. Similar features can be used as desired in the practice of this invention to help stabilize the patch across the entire area of the skin, as disclosed in U.S. Pat. Nos. 6,050,988 and 5,312,456, which are incorporated in their entirety herein by reference.

As exemplified by delivery device 10 shown in FIG. 3, the microprojection array 12 generally comprises a screen having precision microprojections 18 and adjacent openings 24. The array 12 is attached to backing 16 by adhesive layer 14, which may also contact the skin through openings 24. As discussed above, these openings do not offer a very effective means for exposing adhesive layer 14 to the skin. Indeed, adhesive layer 14 provides little retention even with openings having a size of up to about 0.2 mm². As the size of the microprojections is reduced and the density increase, the size of the openings is reduced, further limiting the adhesion effect.

By using backing 14 with adhesive 16 having a larger diameter than array 12, a peripheral adhesive area 26 is formed. While this does improve skin retention of the device, it does not act in a uniform manner. Specifically, areas of the array adjacent the periphery are subject to good retention, but portions of the area towards the center do not have good retention. This lack of uniform retention makes it difficult to obtain reproducible and controlled agent delivery.

Additionally, conventional arrays etched from a single flat sheet cannot bend simulaneously in different directions without wrinkling. As such, such arrays present poor conformation to the skin and cannot configure optimally to domed surfaces, especially when used in ambulatory situations, on curved body parts or on small children.

The problems of poor compliance, and unsatisfactory and non-uniform retention, are exacerbated when the array is greater than about 1 cm².

To overcome the noted limitations of prior art systems, the transdermal agent delivery devices of the invention generally comprise a microprojection array having voids that increase adhesive contact and provide multidirectional flexibility. According to the invention, the voids can comprise various shares and can be oriented in various configurations.

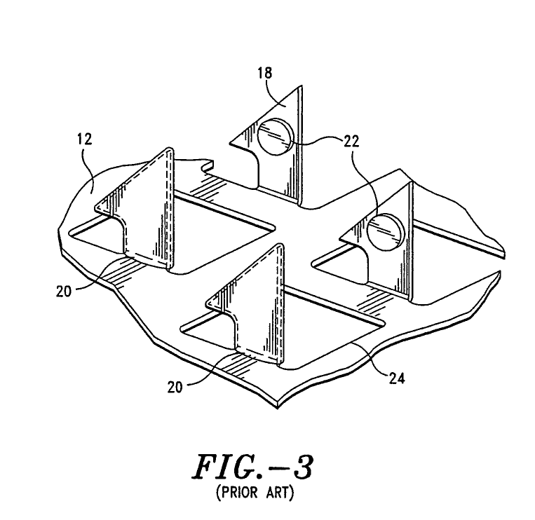

Referring first to FIG. 4, there is shown one embodiment of the invention. As illustrated in FIG. 4, the delivery device 30 includes a microprojection array 32 having at least one void 34. The array 32 is attached to a backing 35 by an adhesive layer 36, shown in cross-section in FIG. 5. In addition to a peripheral adhesive area 38, increased access to adhesive layer 36 is available through void 34. Thus, delivery device 30 presents improved overall retention as well as greater uniformity of retention across the working area of array 32.

An alternative embodiment of the invention is shown in FIG. 6. In the noted embodiment, array 32 is separated into two subunits and void 34 is configured as a ring.

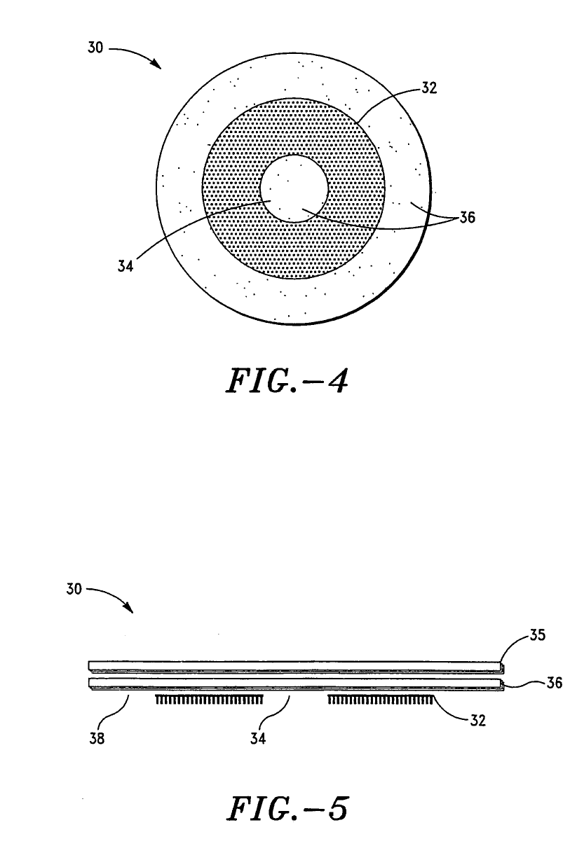

Other embodiments of the invention are shown in FIGS. 7-9, wherein the array 32 includes multiple voids 34. Preferably, the embodiments shown in FIGS. 4-9 are suitable for arrays up to about 5 cm². Also preferably, void 34 has a minimum surface area of 3 mm² and the size of the void is at least approximately 2 mm as measured across its widest length. More preferably, the void is substantially circular. However, according to the invention, other shapes can be employed.

According to the invention, the total void area is in the range of approximately 5-50% of the total area occupied by the array. More preferably, the total void area is in the range of approximately 10-30% of the total area occupied by the array, as shown in FIGS. 4-9.

Specific examples of the noted embodiments include arrays 32 having an area of 3 cm² and a diameter of about 2 cm. Referring to FIG. 4, there is shown an example of void 34 with a diameter of 0.7 cm, a void area of 0.39 cm² and ratio of void area to array of approximately 14.9%. In an alternate example, the void 34 has a diameter of 0.49 cm, a void area of 0.18 cm² and ratio of void area to array of 6.6%.

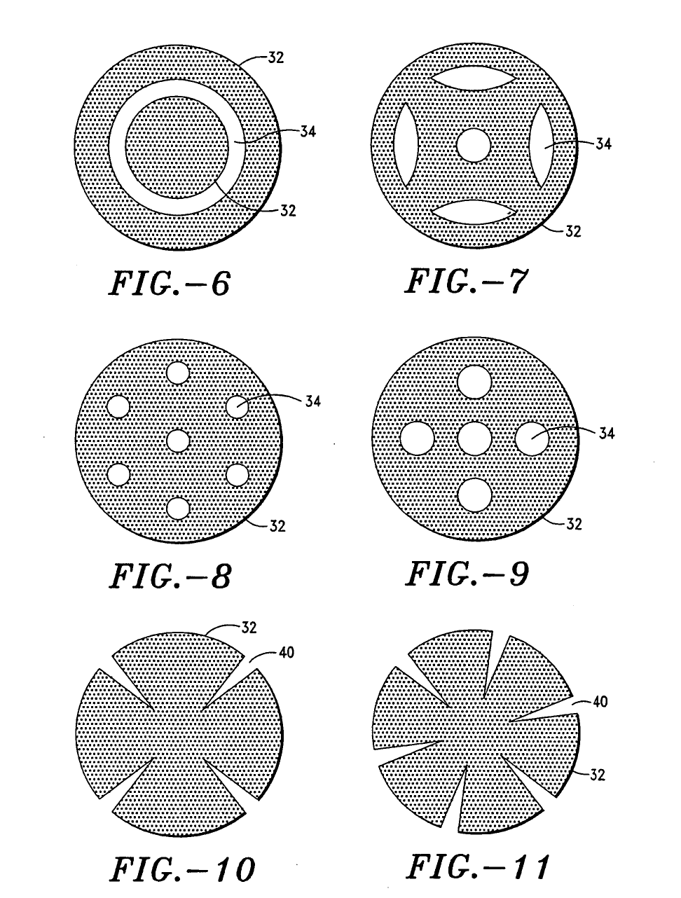

In the configuration shown in FIG. 8, seven (7) voids are shown having diameters of 0.24 cm, a void area of 0.33 cm², and ratio of void area to array of 12.3%. In the configuration shown in FIG. 9, five (5) voids are shown having diameters of 0.33 cm, a void area of 0.43 cm² and ratio of void area to array of 16.9%.

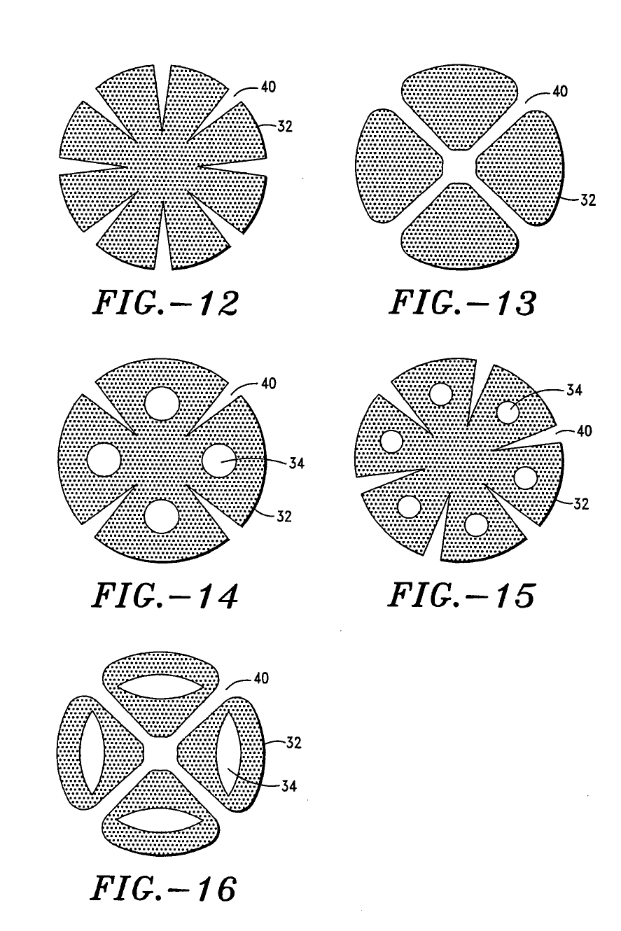

In a further aspect of the invention, arrays larger than about 3 cm² preferably include additional features configured to improve compliance to the skin. In particular, such embodiments are able to accommodate changes in skin doming. FIGS. 10-16 show alternative embodiments of the invention wherein microprojection array 32 is divided by radial voids 40 that extend from a central portion of the array to the periphery. The shown radial fragmentation allows the array to conform to skin doming. In the embodiments shown in FIGS. 13 and 16, the radial voids 40 are joined to divide array 32 into discrete subunits.

In addition to improving compliance of array 32, radial voids 40 present additional areas of attachment to the skin by allowing access to adhesive layer 36. Further, the embodiments shown in FIGS. 14-16 have additional voids 34 to increase the amount of adhesive contact available to the skin to improve retention.

In the noted embodiments, the backing is preferably configured to allow some deformation under low stress to match the improved compliance of the array 32. As such, in preferred embodiments, the backing material comprises a thin (typically less than 0.1 mm) flexible sheet consisting of polymeric material. Additionally, the backing material preferably is capable of linear extension of more than approximately 100 μm/cm in a direction generally parallel to the body surface being pierced. According to the invention, the backing can also be perforated to enhance compliance to the skin.

Suitable backing materials include, without limitation, polyethylene, polyurethane, neoprene, natural rubber, SBR, butyl, butadiene, nitrile, EPDM, ECH, polystyrene, polyester, polyether, polypropylene, EVA, EMA, metallocene resin, PVC, and like materials and blends thereof.

According to the invention, the microprojection array 32 can be manufactured from various metals, such as stainless steel, titanium, nickel titanium alloys, or similar biocompatible materials. Preferably, the microprojection array 32 is manufactured out of titanium.

In another aspect of the invention, the microprojection arrays 32 can also be constructed out of a non-conductive material, such as a polymer. Alternatively, the microprojection member 10 can be coated with a non-conductive material, such as Parylene.

Microprojection arrays having one or more voids as described above exhibit improved retention and more reproducible delivery. In addition, delivery devices embodying features of the invention are more comfortable to the user due to the improved conformation to the skin. Moreover, the features of this invention allow the use of larger arrays, such as arrays having a total area in the range of approximately 5-10 cm², or larger.

An additional aspect of the invention is that designing a microprojection array with one or more voids, as discussed above, allows a reduction in the size of the peripheral adhesive area necessary to attach the device. Specifically, distributing the points of attachment throughout the patch by providing voids to access the adhesive layer enables a reduction in diameter of the peripheral adhesive area.

As discussed above, the microprojection arrays of the invention preferably include an agent-containing coating. When the array is applied to the skin, the microprojections pierce the stratum corneum. Once exposed to interstitial fluids, the agent-containing coating dissolves and the agent is delivered to the surrounding tissue.

Examples of active agents that can be delivered using the microprojection arrays of the invention include ACTH (1-24), BNP, calcitonin, desmopressin, LHRH, LHRH analogs, goserelin, leuprolide, PTH, PYY, vasopressin, deamino [Val4, D-Arg8] arginine vasopressin, buserelin, triptorelin, interferon alpha, interferon beta, interferon gamma, FSH, EPO, GM-CSF, G-CSF, IL-10, glucagon, VEGF, growth hormone releasing factor (GRF) and analogs of these agents including pharmaceutically acceptable salts thereof.

Preferably, the active agent for coating the microprotrusions is selected to have sufficient potency to be therapeutically effective when administered transdermally in an amount of less than about 1 mg, and preferably less than about 0.25 mg, of active agent.

Suitable antigenic agents that can be delivered in accordance with the invention include, without limitation, vaccines, including protein-based vaccines, polysaccharide-based vaccine and nucleic acid-based vaccines, viruses and bacteria.

Further suitable antigenic agents include antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Additional commercially available vaccines, which contain antigenic agents, include, without limitation, flu vaccines, lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. In addition, in certain embodiments of the invention, the nucleic acid can be coupled with a proteinaceous agent or can include one or more chemical modifications, such as, for example, phosphorothioate moieties.

In addition, in the case of DNA, promoter and polyadenylation sequences can also be incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa—3H₂O (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukin 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used. Other adjuvants include heat-shock proteins (HSPs); GTP-GDP; Loxoribine, MPL®; Murapalmitine; and Theramide™. Adjuvants are preferably non-irritating and non-sensitizing.

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitdis, pseudomonas aeruginosa, streptococcus pneumoniae, treponema pallidum, and vibrio cholerae, and mixtures thereof.

The noted antigenic agents or vaccines can be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

Suitable formulations for coating microprojections and means for applying such coatings are disclosed in U.S. patent application Ser. Nos. 10/637,909, 10/608,304, 10/674,626, 10/884,603 and 10/880,702, which are incorporated by reference herein in their entirety.

### EXAMPLE 1

Titanium microprojection members of the type illustrated in FIG. 1 and FIG. 4 are used. Both arrays have an area of 3 cm². The array illustrated in FIG. 4, has a void with a diameter of 0.7 cm and a void area of 0.39 cm². The triangularly shaped microprojections have a length of 150 μm, a tip angle of 60° and a microprojection density of 300 microprojections/cm². The arrays are adhered to the middle portion of a low density polyethylene (LDPE) sheet (5 cm²) having an adhesive film on the skin proximal side of the LDPE sheet between sheet and microprojection member as illustrated in FIG. 1 and FIG. 4.

For testing, the systems are applied to the flanks of hairless guinea pigs (HGPs) using a spring-loaded impact applicator. Following application of the system, the HGPs are housed individually in cages for up to 24 hours. At various time points after application (5 s, 1 h, 4 h, and 24 h), three of the HGPs have their systems removed and system retention is visually evaluated during removal of the system. Results demonstrate that the best system retention is achieved with the system illustrated in FIG. 4.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

It is claimed:

1. A device for transdermally delivering a pharmacologically active agent, the device comprising:
a member having a plurality of stratum corneum-piercing microprojections;
at least one active agent adapted to be delivered transdermally by the microprojections; and
at least one void in the member that allows an adhesive backing to communicate through the void.

2. The device of claim 1, wherein the device includes an adhesive backing that allows the adhesive backing to communicate though the at least one void.

3. The device of claim 1, wherein the active agent is coated on at least one of the microprojections.

4. The device of claim 1, wherein the void enables a reduction in the peripheral adhesive area outside the member.

5. The device of claim 1, wherein the void increases the access of an adhesive backing enabling increased uniformity of adhesion across the member.

6. The device of claim 1, wherein the member has substantially uniform adhesion characteristics across the surface of the member.

7. The device of claim 1, wherein the void is substantially circular.

8. The device of claim 1, wherein the void has a total area greater than approximately 3 cm².

9. The device of claim 1, wherein the void has a surface area greater than approximately 0.2 mm².

10. The device of claim 1, wherein the void is at least approximately 2 mm as measured across its widest length.

11. The device of claim 1, wherein the member has a plurality of voids.

12. The device of claim 1, wherein the member has a total void area in the range of approximately 5-50% of the total member area.

13. The device of claim 1, wherein the member has a total area greater than approximately 1 cm².

14. The device of claim 1, wherein the member has at least one radial void that extends from a central portion of the member to the periphery.

15. The device of claim 1, wherein the void divides the member into at least two subunits.

16. The device of claim 1, wherein the member is separated into two subunits and the void is configured as substantially circular.

17. The device of claim 1, wherein the member has an area of about 3 cm² and a diameter of about 2 cm.

18. The device of claim 1, wherein the void has a diameter of about 0.7 cm, a void area of about 0.39 cm² and ratio of void area to member of approximately 14.9%.

19. The device of claim 1, wherein the void has a diameter of about 0.49 cm, a void area of about 0.18 cm² and ratio of void area to member of approximately 6.6%.

20. The device of claim 1, wherein the member has a plurality of voids having diameters of about 0.24 cm, a void area of about 0.33 cm² and ratio of void area to member of approximately 12.3%.

21. The device of claim 1, wherein the member has a plurality of voids having diameters of about 0.33 cm, a void area of about 0.43 cm² and ratio of void area to member of approximately 16.9%.

22. The device of claim 2, wherein the backing comprises a thin flexible sheet consisting of polymeric material that is optionally perforated.

23. The device of claim 1, wherein the member has a plurality of stratum corneum-piercing microprojections.

24. The device of claim 1, wherein said array is manufactured from a metal consisting of the group of stainless steel, titanium, nickel titanium alloys, or similar biocompatible materials.

25. The device of claim 1, wherein said coating comprises an antigenic agent.

26. A device for transdermally delivering a pharmacologically active agent, the device comprising:
a member having a plurality of stratum corneum-piercing microprojections;
at least one active agent coated on at least one of the microprojections;
at least one void in the member; and
an adhesive backing that allows the adhesive backing to communicate with a subject through the at least one void enabling increased uniformity of adhesion across the member.

27. A method of transdermally delivering a pharmacologically active agent, the method comprising:
providing a delivery system having a microprojection member that includes a plurality of stratum corneum-piercing microprojections, at least one active agent adapted to be delivered transdermally by the microprotrusions, an adhesive backing, and at least one void in the member to allow the adhesive backing to communicate through the void; and
applying the member to a surface so that the adhesive backing communicates with the surface through the void.

28. The method of claim 27, wherein the active agent is coated on at least one microprojection.
